(19) United States
(12) Patent Application Publication
(10) Pub. No.: US 2009/0099536 A1
(43) Pub. Date: Apr. 16, 2009

(54) Bidirectional Phacoemulsification Needle Tips for Torsional and Longitudinal Motion

(76) Inventor: Takayuki Akahoshi Akahoshi, Tokyo (JP)

Correspondence Address: LAW OFFICES OF JERRY A. SCHULMAN, 1S376 SUMMIT AVENUE, COURT C, OAKBROOK TERRACE, IL, 60181, US

(21) Appl. No.: 11/935,409

(22) Filed: Nov. 6, 2007

Related U.S. Application Data
(60) Provisional application No. 60/864,593, filed on Nov. 6, 2006.
(60) Provisional application No. 60/864,955, filed on Nov. 8, 2006.

(51) Int. Cl. A61M 5/32

(52) U.S. Cl. 604/272

(57) ABSTRACT

Tips for phacoemulsification needles feature geometry that allows each tip to cut or emulsify bidirectionally when used torsionally, when used longitudinally or, in some cases, when used both torsionally or longitudinally.

### PRIORITY

Priority is claimed form U.S. Patent Application Ser. No. 60/864,593, filed Nov. 6, 2006 and U.S. Patent Application Ser. No. 60/864,955, filed Nov. 8, 2006, both of which are incorporated herein by reference.

### FIELD OF THE INVENTION

This disclosure relates to surgical instruments and surgical techniques used in eye surgery and, more particularly, to phacoemulsification needle tip designs for use with handpieces that produce torsional motion, linear motion, or both.

### BACKGROUND OF THE INVENTION

A common opthalmological surgical technique is the removal of a diseased or injured lens from the eye. Earlier techniques used for the removal of the lens typically required a substantial incision to be made in the capsular bag in which the lens is encased. Such incisions were often on the order of 12 mm in length.

Later techniques focused on removing diseased lenses and inserting replacement artificial lenses through as small an incision as possible. It is now a common technique to take an artificial intraocular lens (IOL), fold it and insert the folded lens through the incision, allowing the lens to unfold when it is properly positioned within the capsular bag. Similarly, efforts have been made to accomplish the removal of the diseased lens through an equally small incision.

One such removal technique is known as phacoemulsification. A typical phacoemulsification tool includes a handpiece to which is attached a hollow needle. Electrical energy is applied to vibrate the needle at ultrasonic frequencies in order to fragment the diseased lens into small enough particles to be aspirated from the eye through the hollow needle. Commonly, an infusion sleeve is mounted around the needle to supply irrigating liquids to the eye in order to aid in flushing and aspirating the lens particles.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small in incision as possible during such surgery is the minimization of leakage of liquid during and after surgery and the prevention of such a collapse.

Phacoemulsification needles and tips are well represented in the prior art. Needles and tips of varying configurations are well known. A particular shape for a tip or needle is often dictated by the type of handpiece with which the needle is to be used.

U.S. Pat. No. 5,725,495 (Strukel et al) teaches and describes a phacoemulsification handpiece, sleeve and tip illustrating a wide variety of tip configurations and needle cross-sectional configurations.

U.S. Pat. No. 6,007,555 (Devine) teaches and describes an ultrasonic needle for surgical emulsification. The needle and its tip are shown in both circular and oval configurations.

U.S. Pat. No. 6,605,054 (Rockley) teaches and describes a multiple bypass port phaco tip having multiple aspiration ports and a single discharge port to infuse liquid into the eye.

U.S. Pat. No. 5,879,356 (Geuder) teaches and describes a surgical instrument for crushing crystalline eye lenses by means of ultrasound and for removing lens debris by suction which demonstrates the use of a sleeve positioned concentric to the needle and having a pair of discharge ports formed thereon.

U.S. Pat. No. 5,645,530 (Boukhny) teaches and describes a phacoemulsification sleeve, one variation of which has a bellows portion attached to a discharge port ring which directs an annular flow of liquid around the needle and into the eye. The use of the bellows is intended to allow the sleeve to absorb spikes in liquid pressure during the operation.

Published U.S. Patent Application No. 2003/0004455 (Kadziauskas) teaches and describes a bi-manual phaco needle using separate emulsification and aspiration needles inserted into the eye simultaneously during surgery.

U.S. Pat. No. 6,077,285 (Boukhny) teaches and describes a torsional ultrasound handpiece configured to impart both longitudinal and torsional motion to a phacoemulsification needle.

United States Published Patent Application 2006/0217672 (Chon) teaches and describes a phacoemulsification tip having a crimped or swaged distal end to increase efficiency during torsional vibration of the phaco needle.

When used herein the terms “longitudinal motion” or “linear motion” describe the movement of the needle back and forth in an axial direction. That is, during longitudinal or linear motion, the needle moves in a direction away from the hand piece, a motion we will refer to as outward motion, and back toward the hand piece, a motion that we will refer to as inward motion. The term “torsional motion” will mean movement that twists or rotates a needle about its own axis, switching back and forth between clockwise and counterclockwise direction.

I have determined that improved results can be achieved using high-speed handpieces that operate either in the torsional or longitudinal modes if the phacoemulsification tip is provided with a particular geometry that allows it to emulsify or “cut” when it is moved in either of two directions. With respect to longitudinal motion, the two directions are inward and outward while with respect to torsional motion the two directions are clockwise and counterclockwise. In these examples, the motions to which reference are made are opposite one another. As an example, inward longitudinal motion is the opposite of outward longitudinal motion while clockwise torsional motion is the opposite of counterclockwise torsional motion.

I have also determined that selected tip geometries will produce enhanced cutting results when used with either torsional or longitudinal motion.

I have also determined that these improved results are attained when at least a portion of the needle tip is larger in cross-sectional area than the cross-sectional area of the needle shaft.

I have also determined that these improved results can be achieved using the straight phacoemulsification needle configuration, one that is favored by a considerable number of doctors.

I have also determined that providing bi-directional cutting capabilities for both longitudinal and torsional motion in a single tip is a particular advantage when using a handpiece that provides both types of motion as desired.

The teachings and disclosures relating to the present invention will also apply in varying degrees, to hand pieces that provide a combination of motions imparted to the phacoemulsification needle that are neither entirely torsional nor longitudinal.

In accordance with these criteria, I have designed a series of tips that are specifically configured to enhance the emulsifying effect created by the handpiece by creating a series of tips that will emulsify or “cut” bidirectionally in either mode or, in some cases, in both modes.

In a first example, the tip is formed with a series of indentations formed proximate the lip of the opening that the indentations providing angled surfaces.

In another example of the present invention, at least one longitudinal rib is formed on the external surface of the tip.

In another example cutting surfaces are formed on the interior of the of the needle tip.

In another example at least one ono-longitudinal rib is formed on the external tip surface.

While the following describes an example or examples of the present invention, it is to be understood that such description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### BRIEF DESCRIPTION OF THE DRAWINGS

These and further aspects of the present invention will be best understood by reference to the accompanying drawings wherein:

FIG. 1 is a drawing showing prior art oval and square shaped phacoemulsification needle tips;

FIG. 2 is a drawing showing several prior art needle cross-sectional configurations;

FIG. 3 is a schematic sectional view of a round, unaltered phacoemulsification tip;

FIG. 4 is a top view of FIG. 3 after crimping;

FIG. 5 is a lateral view of the tip of FIG. 3 with forming slits cut therein;

FIG. 6 is a top view of FIG. 5;

FIG. 7 is a lateral view of FIG. 5 showing selected crimp points;

FIG. 8 is a top view of FIG. 7;

FIG. 9 is lateral view of FIG. 7;

FIG. 10 is a top view of FIG. 9;

FIG. 11 is a lateral view of FIG. 9 showing second selected crimp points;

FIG. 12 is a top view of FIG. 11;

FIG. 13 is a lateral view of the tip of FIG. 12 after crimping;

FIG. 14 is a top view of FIG. 13;

FIG. 15 is an enlarged view of detail A in FIG. 14;

FIG. 16 is a lateral view of the tip of FIG. 3 showing an angled slit cut therein;

FIG. 17 is a top view of FIG. 16;

FIG. 18 is a lateral view of FIG. 16 showing selected crimp points;

FIG. 19 is a top view of FIG. 18;

FIG. 20 is a lateral view of FIG. 18 after crimping;

FIG. 21 is a top view of FIG. 20;

FIG. 22 is an enlarged view of detail D of FIG. 21;

FIG. 23 is a top view of FIG. 20;

FIG. 24 is a top view of FIG. 20 showing tip segments folded into an approximately 90° angle;

FIG. 25 is a lateral view of another example of a phaco tip having vanes formed thereon;

FIG. 26 is a top view of FIG. 25;

FIG. 27 is a partial schematic sectional view of the tip of FIG. 3 showing a pair of external ribs formed thereon;

FIG. 28 is a top view of FIG. 27;

FIG. 29 is a partial schematic sectional view of a tip with four external ribs formed thereon;

FIG. 30 is a top view of FIG. 29;

FIG. 31 is a partial schematic sectional view showing a tip with six ribs formed thereon;

FIG. 32 is a top view of FIG. 31;

FIG. 33 is another example of a phaco tip with an internal spiral;

FIG. 34 is an end view of FIG. 33;

FIG. 35 is another example of a phaco tip having two cylindrical sections;

FIG. 36 is an end view of FIG. 35;

FIG. 37 is a cross-sectional view of a needle tip having a triangular cross-section;

FIG. 38 is a cross-sectional view of a needle tip having a square cross-section;

FIG. 39 is a cross-sectional view of a needle tip having a pentagonal cross-section;

FIG. 40 is a cross-sectional view of a needle tip having a octagonal cross-section;

FIG. 41 is a first example of a beveled tip;

FIG. 42 is a second example of a beveled tip;

FIG. 43 is a third example of a beveled tip;

FIG. 44 is an end view of another example of the present invention;

FIG. 45 is a lateral view of FIG. 44;

FIG. 46 is an end view of another example of the present invention;

FIG. 47 is a lateral view of FIG. 46;

FIG. 48 is an end view of another example of the present invention;

FIG. 49 is a lateral view of FIG. 48;

FIG. 50 is an end view of another example of the present invention;

FIG. 51 is a lateral view of FIG. 50;

FIG. 52 is a bottom view of FIG. 51;

FIG. 53 is a detail of FIG. 51 showing the bidirectional cutting surfaces in the torsional mode;

FIG. 54 is a bottom view of FIG. 53;

FIG. 55 is a detail of FIG. 51 showing the surfaces used for bilateral cutting in the longitudinal mode; and

FIG. 56 is a bottom view of FIG. 55.

### DETAILED DESCRIPTION OF THE DRAWINGS

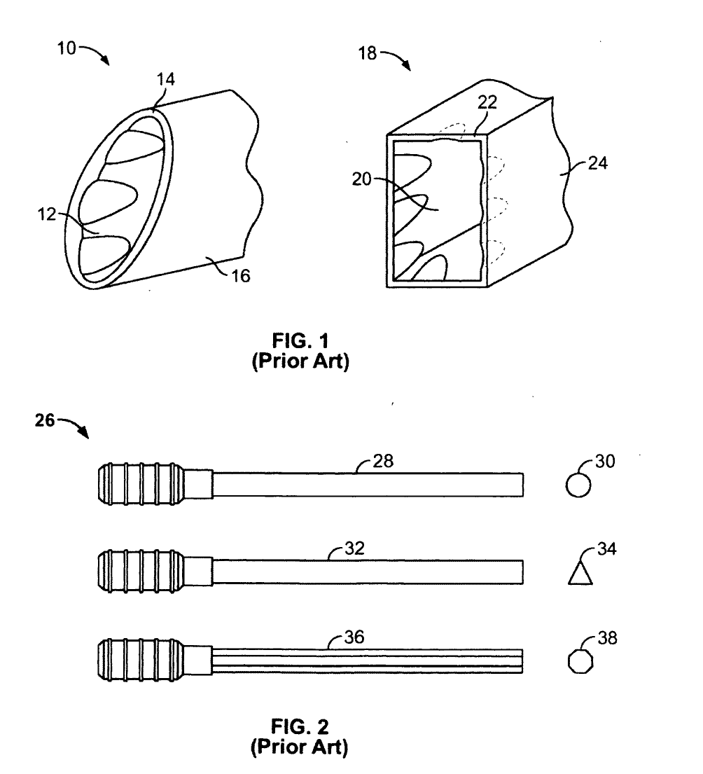

Referring now to FIG. 1, the numeral 10 indicates generally a prior art phacoemulsification needle as shown in U.S. Pat. No. 6,007,555. Needle 10 terminates in a mouth 12 defined by a lip 14 at the end of the needle body 16 with lip 14 and needle body 16 formed with an oval cross-sectional configuration.

Referring to FIG. 1, the numeral 18 indicates generally a prior art phacoemulsification needle tip from U.S. Pat. No. 6,007,555 having a mouth 20 defined by a lip 22 at the end of needle body 24. The cross-sectional configuration of needle 18 at mouth 20 is a rectangle.

Referring now to FIG. 2, the numeral 26 identifies several prior art phacoemulsification needles as described in U.S. Pat. No. 5,725,495, with needle 28 having a circular cross-section as shown at 30, needle 32 having a triangular cross-section as shown at 34 and needle 36 having an octagonal cross-section as shown at 38. Such needles have not, heretofore, been used with handpieces that move in a torsional rather than linear direction.

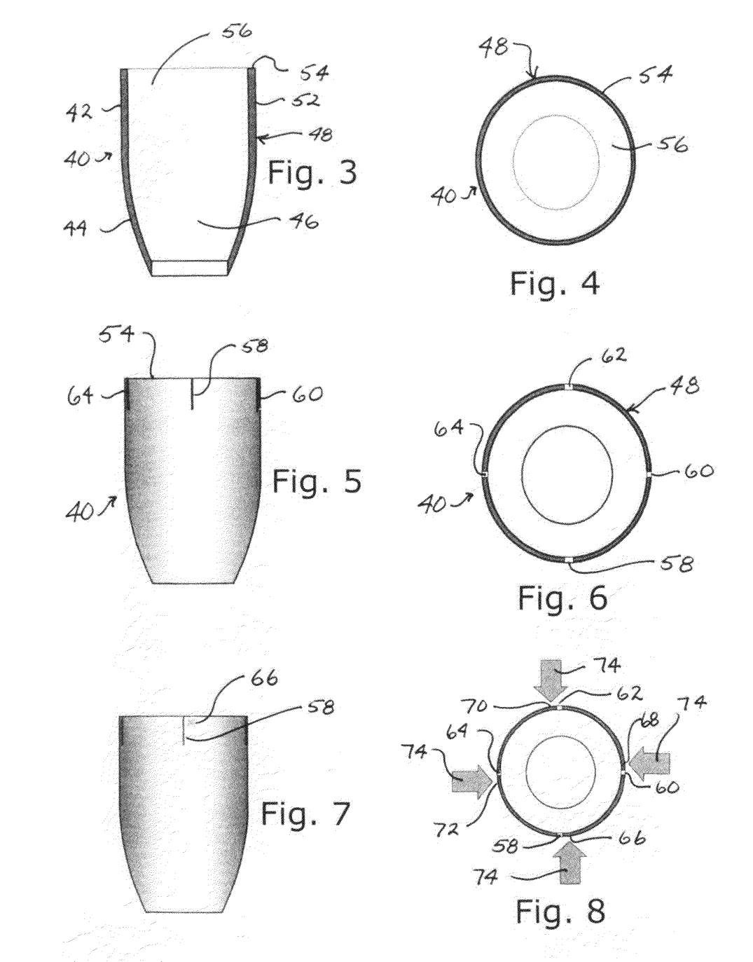

Referring now to FIG. 3, the numeral 40 identifies a standard straight phacoemulsification needle tip having a first, right-circular section 42 and a tapered throat section 44 forming a throat 46 which ends at the point at which tip 40 extends from the hollow phaco needle shaft. Tip 40 has a tip wall 48 having an inner wall surface 50 and an outer wall surface 52.

As seen in FIG. 4, tip wall 48 terminates in a lip 54 which defines a circular opening or mouth 56 of tip 40.

Referring now to FIGS. 5 and 6, a first manufacturing step in the production of a first example of the present invention is shown. Tip 40 has formed thereon a series of axially extending slits 58, 60, 62 and 64. As seen in FIG. 6, slits 58, 60, 62 and 64 are spaced equidistantly around the circumference of wall 48. As seen in FIG. 5, slits 58, 60, 62 and 64 are formed beginning at lip 54 and extending in an axial direction toward throat section 44.

Referring now to FIG. 7, a second step in the manufacturing process is shown wherein a crimping point 66 has been identified which, in this example, is to the right of slit 58. As seen in FIG. 8, a series of crimping points 66, 68, 70 and 72 are formed proximate, respectively, slits 58, 60, 62 and 64. Arrows 74 indicate the direction of the application of a crimping force applied by a die or other crimping means.

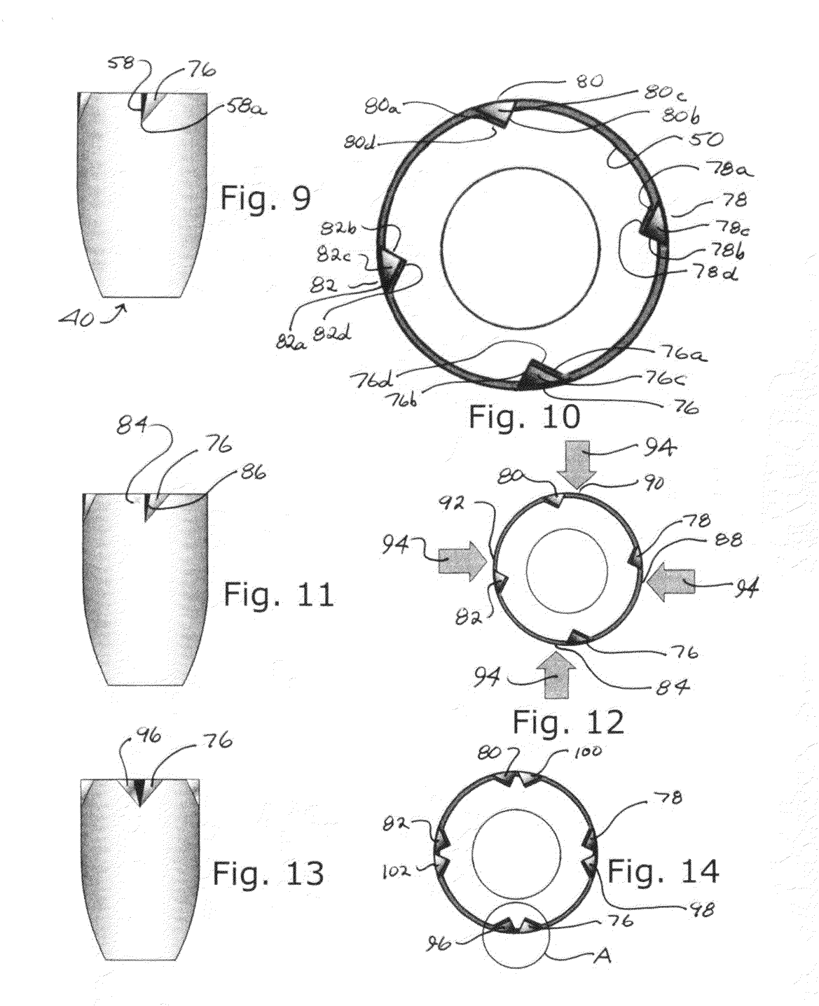

Referring now to FIG. 9, the numeral 76 identifies a completed blade or prong formed at slit 58 after the application of the crimping forces indicated in the directions of arrows 74 in FIG. 8.

Referring now to FIG. 10, blade 76 is shown as having a first upper edge 76a formed as a part of lip 54, a second descending edge 76b which extends downward from edge 76 and terminates proximate end 58a of slit 58 (shown in FIG. 9). Blade 76 also has an outer face 76c formed as a portion of outer tip surface 52 and an inner face 76d formed as a portion of inner tip surface 50.

As seen in FIGS. 8 and 10, blades 78, 80 and 82 are formed with a similar geometry by the foregoing process. Blades 78, 80 and 82 have, respectively, first upper edges 78a, 80a, 82a all formed as a part of lip 54, second descending edges 78b, 80b and 82b outer faces 78c, 80c and 82c, all formed as a portion of outer tip surface 52 and inner faces 78d, 80d and 82d all formed as a portion of inner tip surface 50.

As seen in FIG. 8, blades 78, 80 and 82 are formed with a similar geometry by the foregoing process.

Referring now to FIG. 11, the numeral 84 identifies a second crimp point proximate blade 76. More particularly, crimp point 84 is proximate edge 86 which is the edge that results at slit 58 when blade 76 is crimped inward.

As seen in FIG. 12, additional crimp points 88, 90 and 92 are identified proximate, respectively, blades 78, 80 and 82. Arrows 94 identify the direction in which the crimping force is to be applied to tip 40 at second crimp points 84, 88, 90 and 92.

Referring to FIG. 13, the tip of FIG. 11 is shown with a second set of blades having been formed thereon by the crimping process described in with respect to FIG. 12. Exemplifying this structure, a second crimp 96 is formed proximate crimp 76. As seen in FIG. 14, blades 98, 100, 102 are formed proximate, respectively, blades 88, 90 and 92. For purposes of description we will refer to blades 76, 78, 80 and 82 as “right hand blades” and blades 96, 98, 100 and 102 as “left-hand blades.”

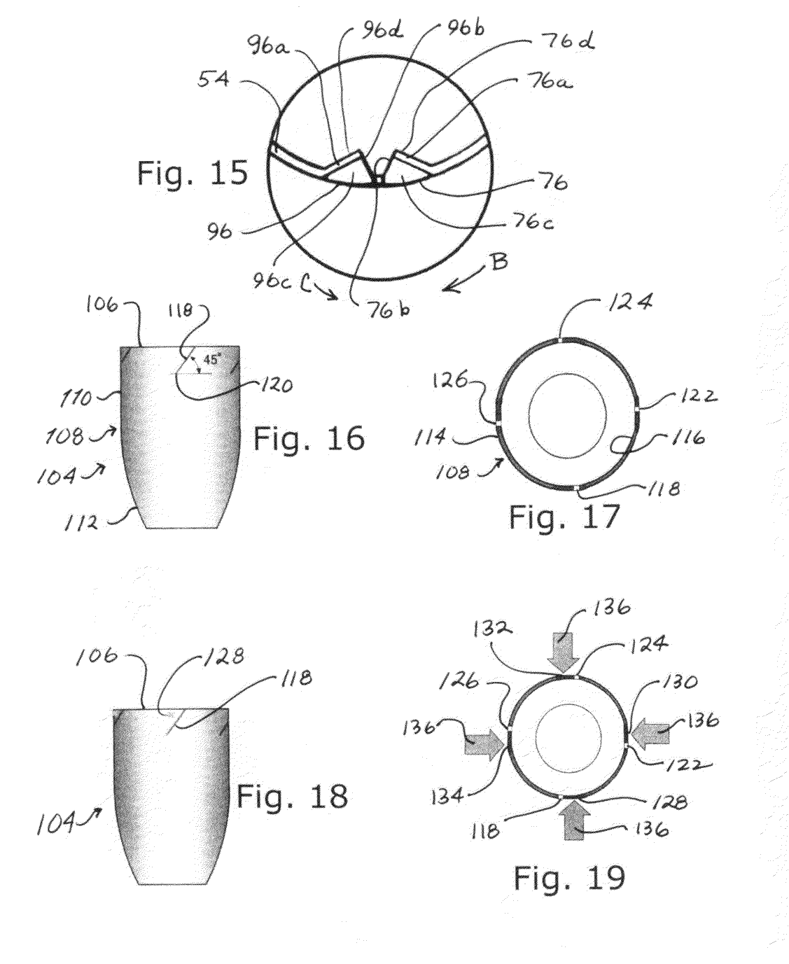

Referring to FIGS. 14 and 15, and in particular to left hand blade 96, the following description will be exemplary of left hand blade 98, 100 and 102. Left hand blade 96 has a first upper edge 96a formed from a portion of lip 54. Left hand blade 96 also has a descending edge 96b extending downward as shown in FIG. 15. Left hand blade 96 also has an outer face 96c forming a portion of outer wall surface 52 and an inner face 96d formed as a portion of inner wall 50.

Tip 40 is intended for use with a torsional hand piece which, as seen in FIG. 15, rotates tip 40 first in a clockwise direction B and then reverses to rotate in a counterclockwise direction C with a frequency to be determined by the particular type of hand piece used and the settings for that hand piece.

As seen in FIG. 15, when the phacoemulsification hand piece is operating in direction B, face 76a and edge 76c form additional emulsifying or cutting surfaces, as does face 96d. When the phacoemulsification hand pieces operating in the counterclockwise or C direction, edge 96b, face 96c and face 76b act as additional emulsifying or cutting surfaces.

In like fashion, when tip 40 is rotated in a clockwise direction, edges 78b, 80b, 82b, faces 78c, 80c and 82c, faces 98a, 100a and 102a all provide additional or cutting surfaces.

When tip 40 is rotated in the counterclockwise direction, edges 98b, 10b, 102b, faces 98c, 100c and 102c, faces 78d, 80d and 82d all act as additional emulsifying or cutting surfaces.

Referring to FIG. 16, the numeral 104 identifies a tip such as that shown in FIG. 3 having a lip 106 and an outer wall 108 having a first, straight section 110 and a second curved throat section 112. As seen in FIG. 17, outer wall 108 has an outer wall surface 114 and an inner wall surface 116.

Referring again to FIG. 16, the numeral 118 identifies a slit cut in straight portion 110 of outer wall 108 and cut at a 45 degree angle to lip 106. In this example, slit 118 terminates on straight section 110 at point 120.

Referring now to FIG. 17, the numeral 118 identifies slit 118 while the numerals 122, 124 and 126 identify similarly-formed slits. Referring now to FIG. 18, the numeral 128 identifies a contact or crimp point proximate slit 118 and lip 106.

Referring now to FIG. 19, point 128 is identified proximate slit 118. Also shown in FIG. 19 are slits 122, 124 and 126. Crimp points 130, 132, and 134 are shown proximate, respectively, slits 122, 124 and 126 as described hereinabove with respect to slit 118 at crimp point 128. Arrows 136 identify the direction in which crimping force is applied to tip 104 to crimp it at points 128, 130, 132 and 134.

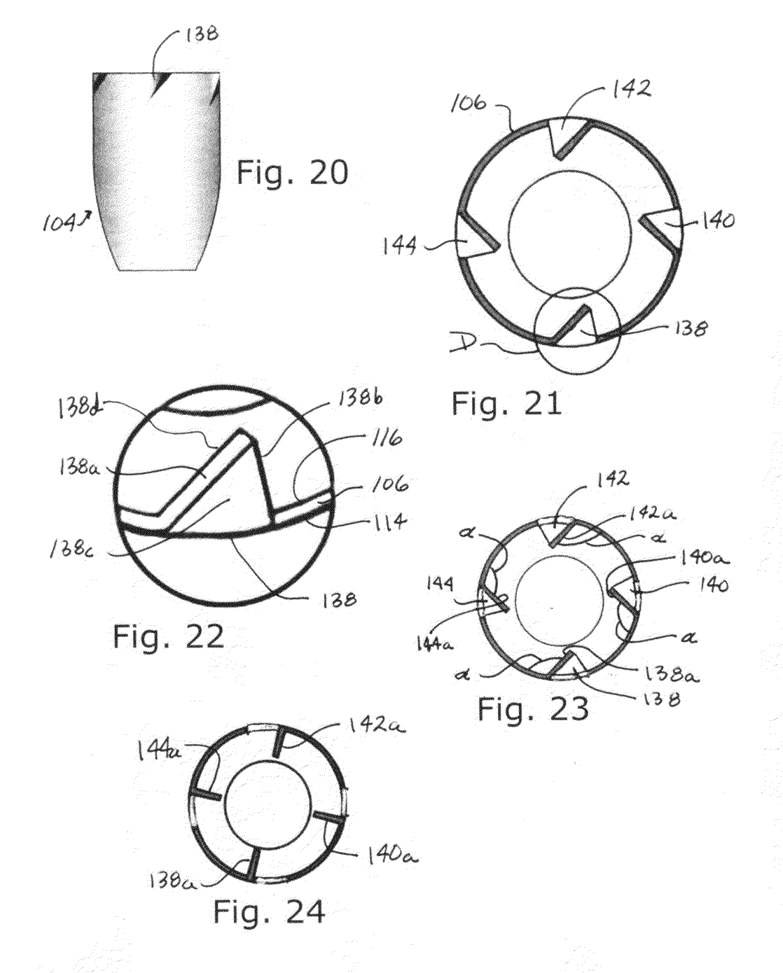

Referring now to FIG. 20, tip 104 is shown with a crimping process completed. As seen in FIGS. 20 and 21, blades 138, 140, 142 and 144 have been formed.

Referring to FIG. 22, blade 138 is shown in enlarged detail from D of FIG. 21. Blade 138 has a top edge 138a which is formed from a portion of lip 106. Blade 138 also has a descending edge 138b formed as part of slit 118. The numeral 138c identifies an outer face of blade 138 formed as a portion of outer wall surface 114 while the reverse side of blade 138 is a rear face 138d formed as a portion of inner wall surface 116.

In this example, blade 138 is exemplary of the remaining blades such that blade 140 also has a top edge 140a, a descending edge 144b a front face 144c and a rear face 144d. In like fashion, blade 146 has a top edge 146a, a descending edge 146b, a front face 146c and a rear face 146d. Blade 148, as well has a top edge 148a, a descending edge 148b, a front face 148c and a rear face 148d.

Referring to FIG. 23 it can be seen that the blades 142, 144, 146 and 148 in this example have been formed inwardly such that each top edge 142a, 144a, 146a and 148a form an acute angle α with lip 106.

Blades such as 142 can be crimped inward to various selected angles. Referring now to FIG. 24, the numeral 146 identifies a tip formed in accordance with the teachings of FIGS. 16-22 with upper edges 138a, 140a, 142a and 144a pressed inwardly to form approximately a right angle with lip 106.

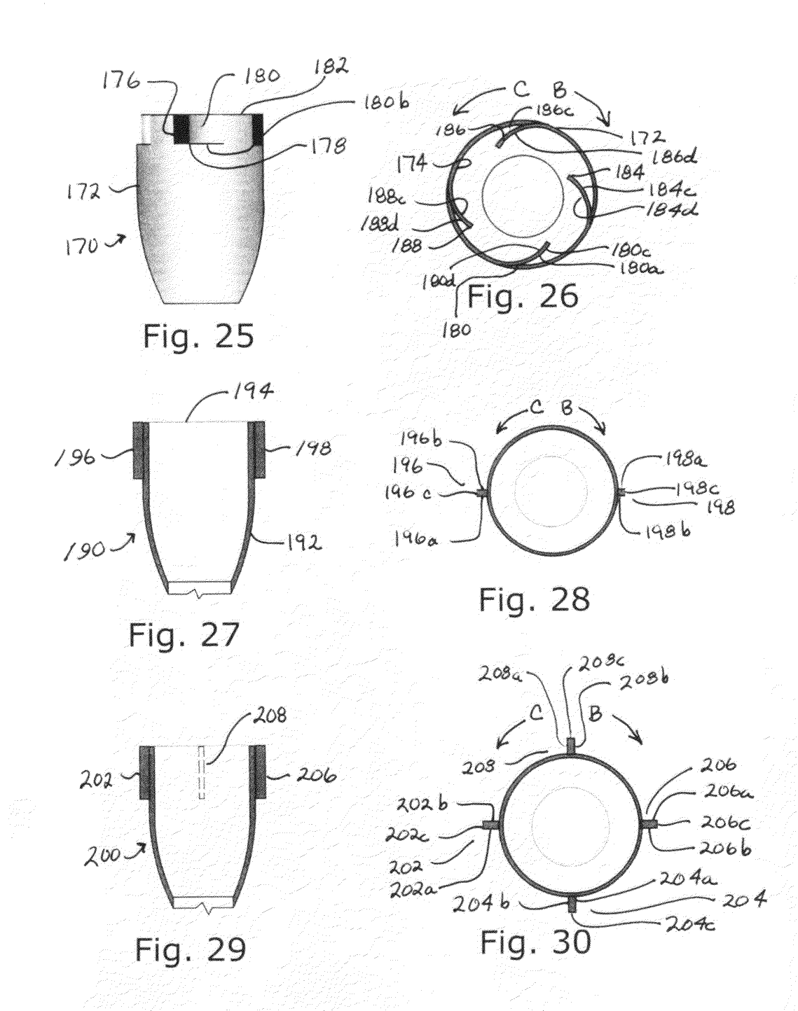

Referring now to FIG. 25, numeral 170 identifies another example of a phacoemulsification tip adapted for effective cutting or emulsification in both the clockwise and counterclockwise direction when used with a torsional hand piece. Tip 170 has an outer wall 172 and as seen in FIG. 26 an inner wall 174. A first, vertical slit is formed through inner and outer walls 172, 174 at 176 of FIG. 25. A second, horizontally extending slit 178 intersects slit 176. A tab or vane 180 formed by slits 176, 178, is pressed or folded inward as described above.

As seen in FIGS. 25 and 26, vane 180, when formed, has an upper edge 180a coextensive with lip 182, a lower edge 180b an outer face 180c and an inner face 180d. As seen in FIG. 26, vane 180 is curved inwardly. In the example shown, vanes 184, 186 and 188 are also formed with each such vane having corresponding surfaces to that as described with respect to vane 180. In particular, vane 184 has an outer face 184c and an inner face 184b, vane 186 has an outer face 186c and an inner face 186d and vane 188 has an outer face 188c and an inner face 188d. As seen in FIG. 26, when tip 170 is rotated in direction B, inner faces 180d, 184d, 186d and 188d act as emulsifying and cutting surfaces, while when tip 170 is rotated in direction C, outer faces 180c, 184c, 186c and 188c act as cutting or emulsifying surfaces. Each vane shown in FIG. 26 has a leading or descending edge 180e, 184e, 186e and 188e which also provides emulsifying or cutting action when tip 170 is rotated in direction C.

Referring now to FIG. 27, the numeral 190 identifies a phacoemulsification tip having an outer wall 192 and a lip 194. In this example, tip 190 has formed thereon first and second external ribs 196, 198 with each such rib extending proximate lip 194 in a direction axially downward. As seen in FIG. 28, rib 196 has a first surface 196a, a second surface 196b disposed opposite surface 196a and a side surface 196c. In like fashion, rib 198 has a first lateral surface 198a, a second lateral surface 198b and a side edge 198c. When tip 190 is rotated in direction B, surfaces 196b and 198b act as emulsifying or cutting surfaces while, when rotated in direction C, rib surfaces 196a and 198a act as emulsifying or cutting surfaces.

Referring now to FIGS. 29 and 30, a tip 200 is shown having four external ribs. As described above, when tip 200 rotates in direction B, surfaces 202b, 204b, 206b and 208b act as emulsifying or cutting surfaces while when rotated in direction C, surfaces 202a, 204a, 206a and 208a act as cutting or emulsifying surfaces.

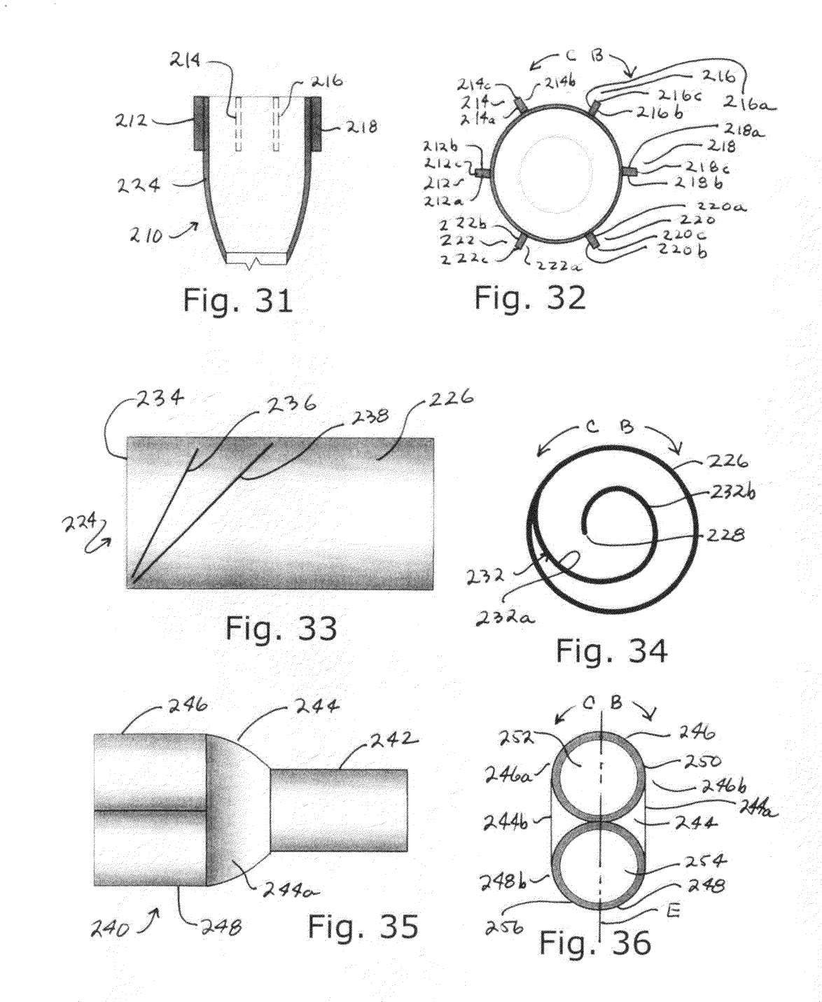

Referring now to FIG. 31, numeral 210 identifies a tip constructed in accordance with the principles described with respect to FIGS. 27 and 28. Tip 210 has six external ribs thereon with ribs 212, 214, 216, 218, 220 and 222 arranged around the periphery of the outer wall 224. Rib 212 has first and second opposed lateral faces 212a and 212b and an external edge 212c; rib 214 has lateral opposed faces 214a and 214b and lateral edge 214c; rib 216 has first and second opposed faces 216a and 216b and lateral edge 216c; rib 218 has opposed lateral faces 218a and 218b and lateral edge 218c; rib 220 has first and second opposed faces 220a, 220b and lateral edge 220c; and rib 222 has first and second opposed faces 222a, 222b and lateral edge 222c.

When tip 210 is rotated in direction B, faces 212b, 214b, 216b, 218b, 220b, and 222b act as emulsifying or cutting surfaces and when rotated in direction C, faces 212a, 214a, 216a, 218a, 220a and 222a act as cutting or emulsifying surfaces.

Referring now to FIGS. 33 and 34, numeral 224 identifies a phacoemulsification tip formed in a spiral configuration. Tip 224 has a wall 226 which, in this example, is formed from a single sheet of rectangular metal such as titanium. Wall 226 is rolled into a spiral configuration as best seen in FIG. 34. Wall 226 has a pair of opposed edges 228, 230 and is rolled in the direction of those edges such that edge 228 is left free within tip 324 and 230 is joined to wall 226 to form a circular tip within which spiral segment 232 is disposed. Spiral segment 232 extends the length of tip 224.

As seen in FIG. 33, tip 224 can be manufactured in a generally right cylindrical configuration which, for reference purposes will be identified as a zero degree bevel. The zero degree bevel indicator in FIG. 33 coincides with lip 234 of tip 224. As a further example, tip 224 may be beveled by severing a portion of tip 224 along the line 236 to produce a 30 degree bevel or along line 238 to produce a 45 degree bevel.

Referring now to FIG. 34, spiral segment 232 may be further described as having a first spiral face 232a and a second spiral face 232b. When tip 224 is rotated in direction B, face 232b becomes a cutting or emulsifying surface while when rotated in direction c, face 232a becomes a cutting or emulsifying surface.

Referring now to FIGS. 34, 35 and 36, numeral 240 identifies a phacoemulsification tip having a first, right circular, segment 242 and an enlarged and tapered throat section, 244, terminating in a pair of tip cylinders 246, 248. Tip cylinder 246 has a mouth 250 defined by lip 252 while tip cylinder 248 has a mouth 254 defined by lip 256. As seen in FIG. 36, tip cylinder 246 has a first substantially semicircular outer wall segment 246a positioned opposite a second substantially semicircular segment 246b as seen in FIG. 36, segment 246a lies to the left of an axis E and segment 246b lies to the right of axis E. In similar fashion, tip cylinder 248 has a first external wall surface 248a lying to the right of axis E and a second semicircular wall surface 248b lying to the left of axis E.

Throat 244 has opposed external surfaces 244a and 244b as shown in FIGS. 35 and 36 with a surface 244a in this example lying to the right of axis E surface 244b lies to the right of axis E.

When tip 240 is rotated in direction B surfaces 246b, 244b, and 248b act as cutting or emulsifying surfaces when while rotating in direction C surfaces 246a, 244a and 248a act as cutting or emulsifying surfaces.

As a feature of the present invention various sizes and shapes of tips and tip blades may be combined on a single tip to provide various cutting parameters and efficiencies. For example, the external ribs in the examples shown in FIGS. 27 through 32 may be applied to the tips shown in FIGS. 11 and 13 if desired.

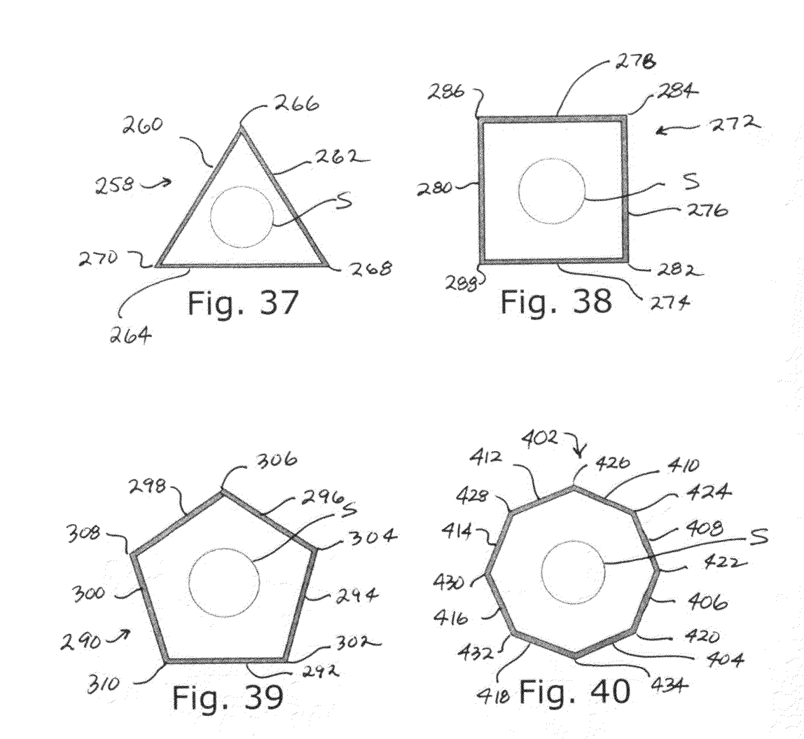

Referring now to FIG. 37 the numeral 258 identifies a phacoemulsification needle tip having a triangular cross-section formed to have a larger cross-section at least one portion thereof than the cross-section of the needle shaft S. Tip 258 has walls 260, 262 and 264 forming lateral edges 266, 268 and 270.

Referring now to FIG. 38 the numeral 272 identifies a phacoemulsification needle tip having a square cross-section formed to have a larger cross-section at least one portion thereof than the cross-section of the needle shaft S. Tip 272 has walls 274, 276, 278 and 280 which form lateral edges 282, 284, 286 and 288.

Referring now to FIG. 39 the numeral 290 identifies a phacoemulsification needle tip having a pentagonal cross-section formed to have a larger cross-section at least one portion thereof than the cross-section of the needle shaft S. Tip 290 has walls 292, 294, 296, 298 and 300, which form lateral edges 302, 304, 306, 308 and 310.

Referring now to FIG. 40 the numeral 402 identifies a phacoemulsification needle tip having an octagonal cross-section formed to have a larger cross-section at least one portion thereof than the cross-section of the needle shaft S. Tip 402 was walls 404, 406, 408, 410, 412, 414, 416 and 418, forming lateral edges 420, 422, 424, 426, 428, 430, 432 and 434.

The lateral edges identified in FIGS. 37-40 form cutting or emulsification surfaces when tips 258, 272, 290 and 402 are rotated in either a clockwise or counter-clockwise direction. It is a feature of the present invention that blades such as those described heretofore may also be formed on tips 258, 272, 290 and 402. It is also a feature of the present invention that the cross-sectional shapes described above are exemplary only and that other such shapes may also be used.

The foregoing examples have been presented with a zero degree bevel. It is also a feature of the present invention that a bevel may be applied to the tips described hereinabove and the ribs, vanes or blades of each tip modified accordingly. For the purposes of this description, the term “bevel” means the angling of the tip proximate the tip opening.

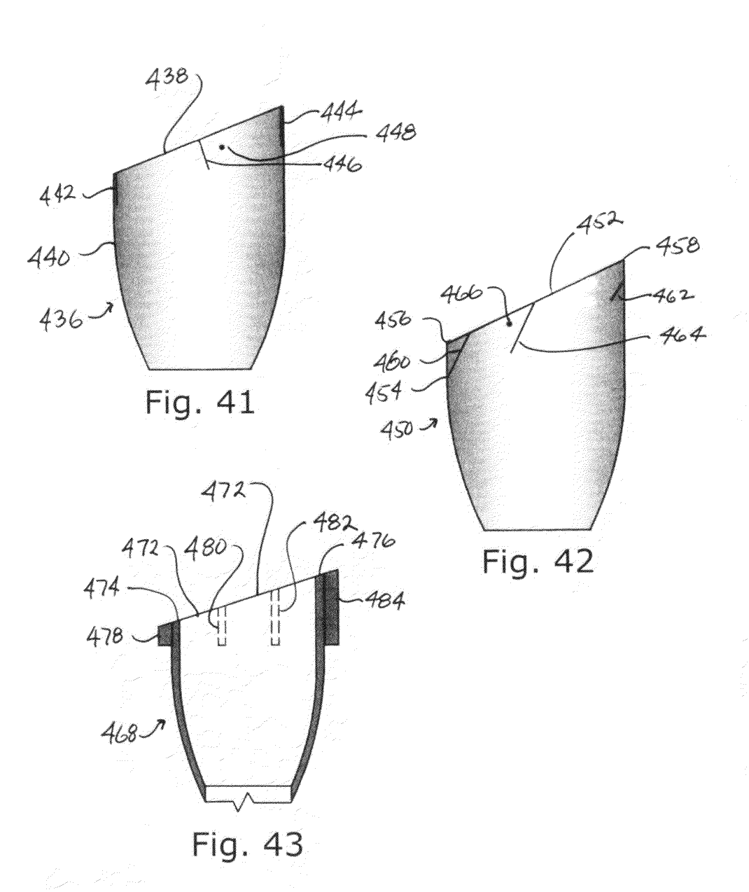

Referring now to FIG. 41 an example of the tip described in FIGS. 3 through 10 is shown with a bevel applied. The numeral 436 identifies a tip having a bevel applied at the end thereof forming a lip 438 and a tip wall 440 that varies in height from a low point 442 to a high point 444. In this example, points 442 and 444 are positioned opposite one another such that an axis drawn through both points would bisect the opening defined by lip 438.

In this example a slit 450 is formed at lip 438 and extends axially from point 442 while a slit 452 is formed at lip 438 and extends axially from point 444. Third and fourth slits are also formed opposite one another beginning at lip 438. One such slit 446 is shown and is formed perpendicular to lip 438 and extends at an angle downward. The remaining slit is formed in the same manner. A crimp point 448 identifies that point at which tip 436 is crimped to produce a blade as described above, it being understood that in this example four such blades are formed. The number of blades can vary as desired to create tips with different operating characteristics.

Referring now to FIG. 42, a tip 450 is shown having a bevel as described above, with a lip 452, a wall 454, a low point 456 and a high point 458. Slits 460, 462 are formed proximate points 456 and 458 respectively, and, in this example, at an angle of about 45° as seen in FIG. 16. Two additional slits are formed, with slit 464 being exemplary thereof. In this example, slit 464 is formed at an angle of 45° with lip 452. A crimp point 466 identifies where tip 450 will be crimped to produce a blade as described above. In this example, four such blades will be formed.

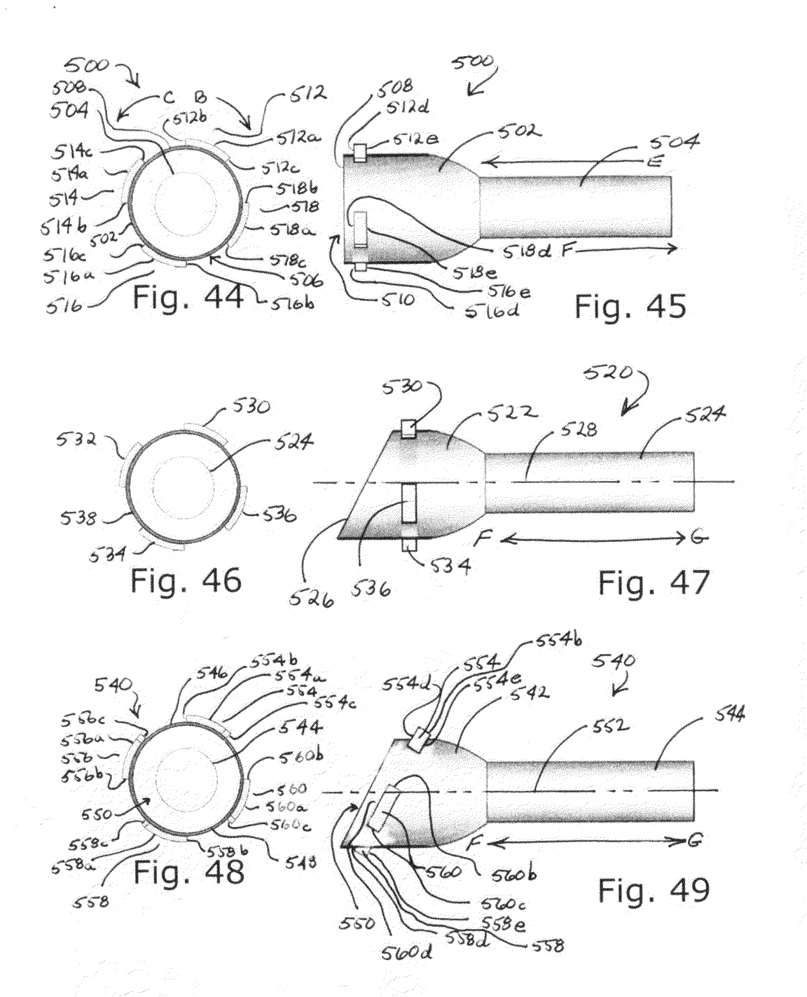

Referring now to FIG. 43 the numeral 468 identifies a tip beveled as described above and having an outer wall 470 terminating at a lip 472. Wall 470 has high and low points 474, 476, respectively and, as shown in FIGS. 31 and 32, a series of ribs formed on wall 470. In this example, six such ribs are formed, with ribs 478, 480, 482 and 484 being exemplary thereof.

As can be seen in this example, rib 478 is shorter than rib 480 which, in turn, is shorter than rib 482 which, in turn, is shorter than rib 484, it being understood that two additional ribs corresponding to ribs 480, 482 are also formed on wall 470, in corresponding, opposite positions.

Referring now to FIGS. 44 and 45, the numeral 500 identifies a phacoemulsification needle having a tip 502 and a hollow needle shaft 504. Tip 502 has an outer wall 506 terminating in a lip 508 which defines a needle tip mouth 510.

As seen in FIG. 44, when used in the torsional mode tip 502 turns in a clockwise direction B and is then reversed to turn in a counterclockwise direction C. As seen in FIG. 45, when needle 500 is used in a longitudinal mode, tip 502 modes in an outward direction E, and an inward direction F.

In the example shown in FIG. 44, tip 502 has four externally formed ribs 512, 514, 516 and 518. As seen in FIG. 44, rib 512 has an outer face 512a and opposed lateral faces 512b and 512c. As seen in FIG. 45, rib 512 also has a front face 512d and a rear face 512e. Similarly, as seen in FIG. 44, rib 514 has a front face 514a and opposed lateral faces 514b and 514c; rib 516 has a front face 516a and an opposed lateral faces 516b and 516c; and rib 518 has a front face 518a and opposed lateral faces 518b and 518c.

As seen in FIG. 45, rib 516 also has front and rear faces 516d, 516e respectively, rib 518 has front and rear faces 518d and 518e, respectively. It should be understood that rib 514 although not shown in FIG. 45, similarly has a front face 514d and a rear face 514e.

Referring now to FIGS. 46 and 47, the numeral 520 identifies a phacoemulsification needle having a tip 522 and a hollow shaft 524. In this example, tip 522 is beveled at an angle of approximately 30°, meaning that lip 526 is formed at a 30° angle with respect to needle axis 528.

In the example shown in FIGS. 46 and 47, tip 522 has four ribs 530, 532, 534 and 536 formed on outer wall 538 equidistantly spaced and formed such that a plane passing through said ribs would be substantially perpendicular to axis 528. For purposes of this example, ribs 530, 532, 534 and 536 are configured substantially the same as ribs 512, 514, 516 and 518 in FIG. 44 and exhibit the same cutting or emulsifying properties.

Referring now to the example in FIG. 44, when tip 502 is rotated in direction B, lateral rib faces 512b, 514b, 516b and 518b act as cutting or emulsifying surfaces.

When the rotation of tip 500 is reversed to direction C, lateral rib faces 512c, 514c, 516c and 518c act as cutting or emulsifying surfaces.

As seen in FIG. 45, when tip 502 is moved in the outward longitudinal direction G, front faces 512d, 514d, 516d and 518d act as cutting or emulsifying surfaces, while, when tip 502 is moved in the inward or F direction, rear faces 512e, 514e, 516e and 518e act as cutting or emulsifying surfaces.

In the example shown in FIGS. 46 and 47, it should be appreciated that corresponding faces on ribs 530, 532, 534 and 536 exhibit the same cutting or emulsifying characteristics consistent with the motion of tip 522.

Referring now to FIGS. 48 and 49, the numeral 540 identifies a phacoemulsification needle having a tip 542 and a hollow shaft 544. Tip 542 has an outer wall 546 and a lip 548 defining a mouth 550. As seen in FIG. 49, tip 542 is beveled meaning that lip 548 is formed at an angle to axis 552. In the example in FIG. 8, the bevel is approximately 30°.

As seen in FIG. 48, tip 542 has four ribs 554, 556, 558 and 560 formed on wall 546. Rib 554 has an outer face 554a. As seen in FIG. 49, rib 554 also has a front face 554d and a rear face 554e in the example shown in FIG. 49, front face 554d is parallel to lip 548. In similar fashion, the front faces 556d, 558d and 560d are parallel to lip 548 and ribs 554, 556, 558 and 560 are formed the same distance from lip 548. When tip 542 is used in a torsional mode in direction C, lateral edge 554b and front face 554d become cutting or emulsifying surfaces. In similar fashion, surfaces 556b, 556d, 558b, 558d, 560b and 560d become cutting or emulsifying surfaces. When tip 542 is rotated in direction D, surfaces 554c, 554e, 556c, 556e, 558c, 558e, 560c and 560e become cutting or emulsifying surfaces.

When tip 542 is used in the longitudinal mode in direction G, faces 554c, 554d, 556c, 556d, 558c, 558d, 560c, 560d become cutting or emulsifying surfaces. When moved in direction F longitudinally, surfaces 554b, 554e, 556b, 556e, 558b, 558e, 560b, and 560e become cutting or emulsifying surfaces.

It should be understood that although the foregoing examples include four such ribs, the number, positioning and shape of such ribs is not to be limited to these examples only. Other rib shapes, other numbers of ribs, other selected spacings of ribs along the outer surface of the phacoemulsification needle tip and other angles for lip 526 may also be selected.

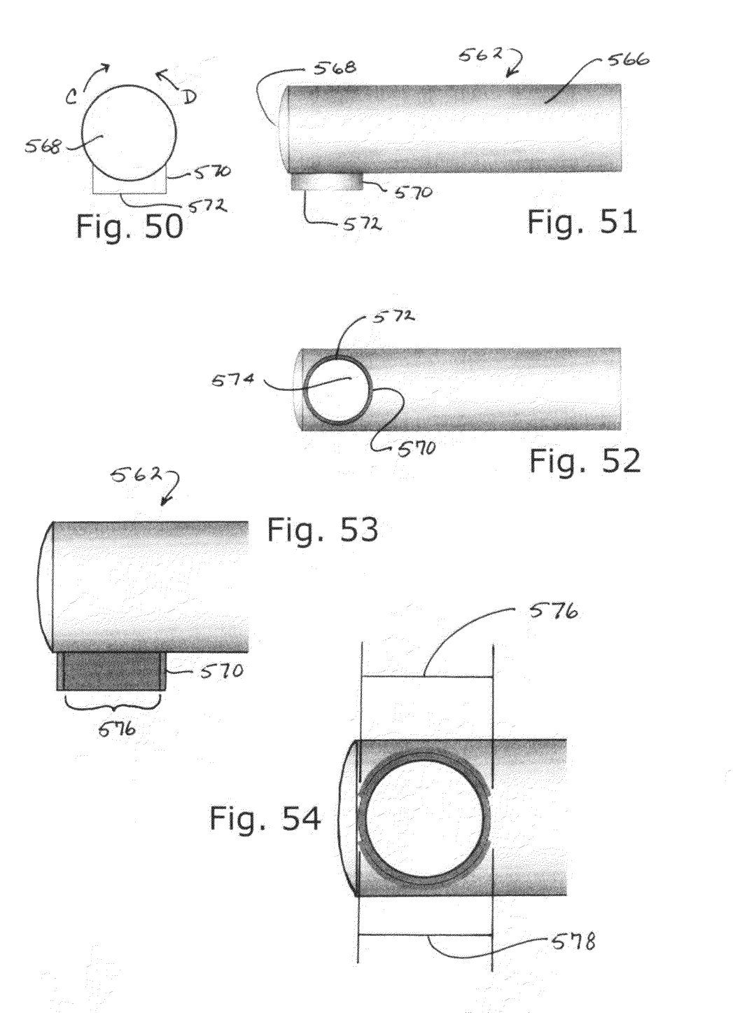

Referring now to FIGS. 50 and 51, the numeral 562 identifies a phacoemulsification needle having a cylindrical needle body 564 with an outer wall 566. Needle 562 terminates in a needle end surface 568 which, in this example, is a solid and arcuate surface.

Needle 562 also has a port collar 570 having a lip 572. In this example, collar 570 is formed as a right circular cylindrical section.

Referring now to FIG. 52, collar 570 and lip 572 define a mouth 574 which communicates to the hollow interior of needle 562. Mouth 574 functions as an aspiration port to remove emulsified particles during phacoemulsification and have them drawn off through the hollow needle shaft 562.

Referring now to FIGS. 53 and 54, the use of needle 562 in the torsional mode is shown. As seen in FIG. 53, a first portion 576 of the outer surface of collar 570, indicated by shading, is shown. As seen in FIG. 54, a second, similar portion 578 is shown. When needle 562 is used in the torsional mode, surface portion 576 forms a cutting or emulsifying surface when rotated in the D direction. In like fashion, surface portion 560 forms a cutting or emulsifying surface when tip 564 is rotated in the C direction.

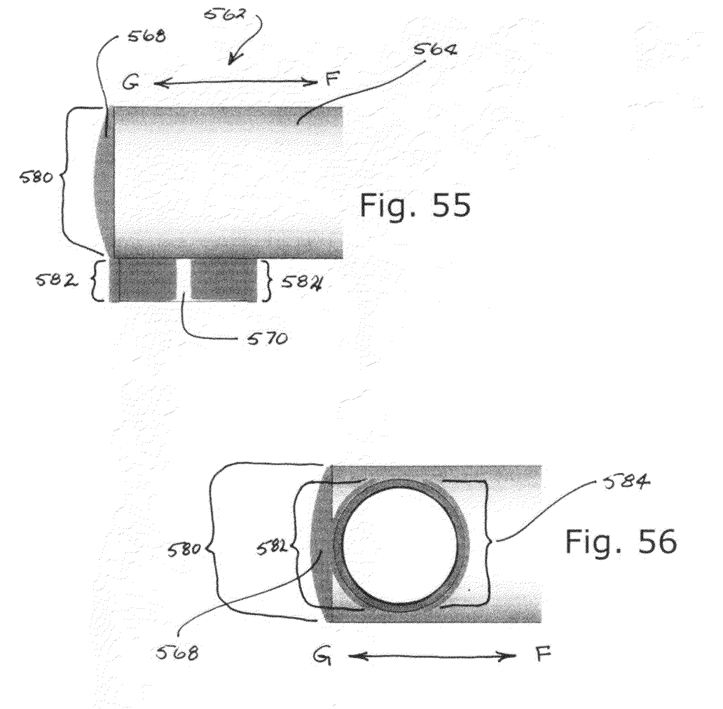

Referring now to FIGS. 55 and 56, an enlarged detail of tip 564 is shown and exemplifies the surfaces used as cutting or emulsifying surfaces when tip 564 is used in the longitudinal mode. As seen in FIG. 55, shaded surface 580 of needle end surface 568 as well as shaded surface 582 of collar 570 act as cutting or emulsifying surfaces when tip 564 is moved in the G direction. In like fashion, when tip 564 is moved in the F direction, shaded surface 584 of collar 570 acts as a cutting or emulsifying surface.

In this fashion, the tips used as examples exhibit cutting or emulsifying action in both directions of rotation when used torsionally and in both directions of movement when used longitudinally.

I claim:

1. A phacoemulsification needle for emulsifying body tissue, said needle adapted to be attached to a phacoemulsification hand piece, said needle comprising:
a hollow needle shaft having a distal end and a proximal end,
said needle shaft having a longitudinally-extending central axis;
means formed at said proximal end to mount said needle to said handpiece;
a hollow needle tip formed at said distal end and communicating with said hollow needle shaft,
said tip having a side wall terminating at a lip,
said side wall and said lip defining a needle mouth,
said tip having at least one cross-section greater in size than the cross-section of said needle shaft;
first means formed on said tip for emulsifying said tissue when said needle is rotated about said axis in a first direction; and
second means formed on said tip for emulsifying said tissue when said needle is rotated about said axis in a second direction.

2. The apparatus as recited in claim 1 wherein said first and second emulsifying means extend into said tip mouth at an angle to said side wall.

3. The apparatus as recited in claim 2 wherein at least one said emulsifying means includes a portion of said lip.

4. The apparatus as recited in claim 3 wherein at least one said emulsifying means is triangular and includes a first edge which emulsifies when said needle is rotated in said first direction and a second edge which said emulsifying means emulsifies when said needle is rotated in said second direction.

5. The apparatus as recited in claim 1 wherein said first and second emulsifying means are formed on said side wall.

6. The apparatus as recited in claim 5 wherein at least one said emulsifying means comprises a rib having first and second surfaces,
said first rib surface acting to emulsify said tissue when said needle is rotated in said first direction and
said second rib surface acting to emulsify said tissue when said needle is rotated in said second direction.

7. The apparatus as recited in claim 6 wherein said first and second rib surfaces are substantially parallel to said needle axis.

8. The apparatus as recited in claim 6 wherein said emulsifying surfaces are formed at an angle to said needle axis.

9. The apparatus as recited in claim 1 wherein said emulsifying means comprises a planar internal spiral.

10. The apparatus as recited in claim 1 wherein said emulsifying means comprises at least first and second hollowing cylindrical segments formed proximate said distal end,
said segments being contiguous to and coextensive with one another,
each said segment having a side wall and a lip,
each said segment having a mouth defined by that segment's sidewall and lip,
each said segment communicating with said hollow needle shaft.

11. A phacoemulsification needle for emulsifying body tissue, said needle adapted to be attached to a phacoemulsification hand piece, said needle comprising:
a hollow needle shaft having a distal end and a proximal end,
said needle shaft having a longitudinally-extending central axis;
means formed at said proximal end to mount said needle to said handpiece;
a hollow needle tip formed at said distal end and communicating with said hollow needle shaft,
said tip having a side wall terminating at a lip,
said side wall and said lip defining a needle mouth,
said tip having at least one cross-section greater in size than the cross-section of said needle shaft;
first means formed on said tip for emulsifying said tissue when said needle is moved longitudinally along said axis in a first direction; and
second means formed on said tip for emulsifying said tissue when said needle is moved longitudinally in a second direction.

12. The apparatus as recited in claim 11 wherein at least one said emulsifying means is formed on said side wall.

13. The apparatus as recited in claim 11 wherein at least one said emulsifying means comprises a rib having first and second emulsifying surfaces,
each said emulsifying surface being formed substantially parallel to said lip.

14. The apparatus as recited in claim 11 wherein said lip is formed at an angle to said needle axis; and
at least one said emulsifying means comprises a rib having first and second emulsifying surfaces,
said first and second emulsifying surfaces formed substantially parallel to said lip.

15. The apparatus as recited in claim 1 wherein said tip is formed with a polygonal cross-section.

16. A phacoemulsification needle for emulsifying body tissue, said needle adapted to be attached to a phacoemulsification hand piece, said needle comprising:
a hollow needle shaft having a distal end and a proximal end,
said needle shaft having a longitudinally-extending central axis;
means formed at said proximal end to mount said needle to said handpiece;
a hollow needle tip formed at said distal end and communicating with said hollow needle shaft, said tip having a side wall terminating at a lip,
said side wall and said lip defining a needle mouth,
said tip having at least one cross-section greater in size than the cross-section of said needle shaft;
first means formed on said tip for emulsifying said tissue when said needle is rotated about said axis in a first direction;
second means formed on said tip for emulsifying said tissue when said needle is rotated about said axis in a second direction;
third means formed on said tip for emulsifying said tissue when said needle is moved longitudinally along said axis in a third direction; and
fourth means formed on said tip for emulsifying said tissue when said needle is moved longitudinally in a fourth direction.

17. The apparatus as recited in claim 16 wherein at least one of said first, second, third or fourth emulsifying means is formed on said side wall.

18. The apparatus as recited in claim 16 wherein at least one said emulsifying means comprises a rib having first and second surfaces,
said first rib surface acting to emulsify said tissue when said needle is rotated in said first direction and
said second rib surface acting to emulsify said tissue when said needle is rotated in said second direction.

19. The apparatus as recited in claim 18 wherein at least one of said first and second rib surfaces is substantially parallel to said needle axis.

20. The apparatus as recited in claim 18 wherein at least one of said first and second rib surfaces is formed at an angle to said needle axis.

21. The apparatus as recited in claim 16 wherein at least one said emulsifying means comprises a rib having third and fourth surfaces,
said third rib surface acting to emulsify said tissue when said needle is moved in said third direction, and
said fourth rib surface acting to emulsify said tissue when said needle is rotated in said fourth direction.

22. The apparatus as recited in claim 21 wherein at least one of said third and fourth rib surfaces is substantially parallel to said needle axis.

23. The apparatus as recited in claim 18 wherein at least one of said third and fourth rib surfaces are formed at an angle to said needle axis.

24. A phacoemulsification needle for emulsifying body tissue, said needle of the type adapted to being attached to a phacoemulsification hand piece, said needle of the type having a hollow needle shaft, said needle shaft having a longitudinally-extending central axis,
said needle comprising:
a first emulsifying surface formed at the distal end of said needle shaft closing off said needle shaft;
a needle port,
said needle port formed on said needle shaft proximate said first emulsifying surface and extending through said needle side wall;
an upstanding collar surrounding said needle port and extending away from said side wall;
a first portion of said collar forming at least one emulsifying surface when said needle is moved in a first longitudinal direction;
a second portion of said collar forming an emulsifying surface when said needle is moved in a second longitudinal direction;
a third portion of said collar forming an emulsifying surface when said needle is rotated about said axis in a first direction; and
a fourth emulsifying surface when said needle is rotated about said axis in a second direction.

25. The apparatus as recited in claim 24 wherein said collar extends about the entire periphery of said port.
